(19) United States
(12) Patent Application Publication
(10) Pub. No.: US 2026/0021229 A1
(43) Pub. Date: Jan. 22, 2026

(54) EXTRACORPOREAL BLOOD TREATMENT MACHINE, COMPUTER-IMPLEMENTED DETECTION METHOD, AND COMPUTER PROGRAM

(71) Applicant: B. Braun Avitum AG, Melsungen (DE)

(72) Inventor: Waldemar Janik, Melsungen (DE)

(21) Appl. No.: 19/268,082

(22) Filed: Jul. 14, 2025

(30) Foreign Application Priority Data
Jul. 16, 2024 (DE) 10 2024 206 668.0

(51) Int. Cl. A61M 1/16; A61M 1/36; A61M 60/113

(52) U.S. Cl. CPC A61M 1/1603; A61M 1/1694; A61M 1/3644; A61M 1/3655; A61M 60/113

(57) ABSTRACT

An machine is used for extracorporeal treatment of blood from a patient. The machine includes a dialyzer, a blood circuit with arterial and venous sections, a dialysis fluid circuit, a blood property and/or dialysate property recording unit, and a control unit. The blood treatment machine is configured to perform priming of the extracorporeal blood circuit with priming fluid, request connection of an arterial shunt section with the arterial section filled with priming fluid by priming and/or to ascertain this connection, request connection of a venous shunt section with the venous section filled with priming fluid by the priming and/or to ascertain this connection, and detect and/or quantify and/or check a recirculation and/or a recirculation rate at the shunt depending on a blood and/or dialysate property. A computer-implemented detection method is used for detecting recirculation at a shunt of the machine. The method can be executed with a computer program.

### CROSS-REFERENCE TO RELATED APPLICATION

This application claims priority under 35 U.S.C. § 119 to German Application No. 10 2024 206 668.0, filed on Jul. 16, 2024, the content of which is incorporated by reference herein in its entirety.

### FIELD

The present disclosure relates to an extracorporeal blood treatment machine, in particular a dialysis machine, for extracorporeal blood treatment, such as hemodialysis, hemofiltration, hemodiafiltration, and/or ultrafiltration, a computer-implemented detection method for detecting recirculation at a shunt of an extracorporeal blood circuit of the blood treatment machine, and a computer program. An extracorporeal blood treatment machine has a dialyzer with a semipermeable membrane for exchanging substances between a patient's blood conveyed in an extracorporeal blood circuit and a dialysis fluid conveyed in a dialysis fluid circuit. The extracorporeal blood circuit has an arterial section, which is intended for connecting an arterial shunt section of a shunt of the patient, and a venous section, which is intended for connecting a venous shunt section.

### BACKGROUND

In an extracorporeal blood treatment, for example blood purification in the form of hemodialysis, hemofiltration, or hemodiafiltration, blood is removed from a dialysis patient via an arterial vascular access and fed to a dialyzer for blood treatment via an extracorporeal blood circuit. A dialysis fluid is also fed to the dialyzer via a separate dialysis fluid circuit. In the dialyzer, blood from the bloodstream and dialysis fluid from the dialysis fluid circuit are brought into contact via a semipermeable membrane, allowing an exchange of substances between the blood and the dialysis fluid to take place. During dialysis treatment for patients with kidney failure, harmful substances can be removed from the blood, as well as excess water that accumulates in the body due to underlying kidney failure, for example. The blood cleaned in this way is then returned to the patient via a venous access.

In extracorporeal blood treatment, it is extremely important that the arterial and venous cannulas are correctly spaced at the patient's shunt, that they puncture the shunt correctly, and that the shunt is free of stenosis, as otherwise recirculation from the venous shunt section to the arterial shunt section may occur. Recirculation means that blood that has already been cleaned is not returned to the vein and thus to the body, but bypasses the body and flows directly into the arterial shunt section, from where it is conveyed back into the dialyzer. Recirculation therefore reduces the efficiency of blood treatment and leads to longer treatment times. It is therefore very important to identify any recirculation that occurs and take measures to remedy it.

The publication DE 10 2021 116 343 A1 discloses a recirculation measurement using two interim circuits with kinetically different diffusion states.

Another method for detecting recirculation at the shunt is disclosed in publication EP 2 783 713 A1. For this purpose, a property of the dialysate that correlates with a blood property of the extracorporeal blood circulation is recorded at the dialysate outlet of the dialyzer. A blood-side bolus administration during dialysis is then used to determine any recirculation based on the dialysate properties recorded at the dialysate outlet.

A disadvantage of the methods according to the prior art is that an explicit bolus administration and/or blood sampling including analysis is required.

### SUMMARY

The purpose of the present disclosure, on the other hand, is to avoid or at least reduce the disadvantages of the prior art and to provide an extracorporeal blood treatment machine, a computer-implemented detection method, and a computer program, through which more efficient and safer extracorporeal blood treatment is provided.

A basic idea of the present disclosure is to provide an extracorporeal blood treatment machine that is set up to check the shunt of a patient prior to extracorporeal blood treatment, especially dialysis, i.e., before or at the start of extracorporeal blood treatment, according to the principle of fluid bolus delivery, for recirculation at the shunt.

Another fundamental idea of the present disclosure is to provide a computer-implemented detection method that is implemented in a control unit of the extracorporeal blood treatment machine, in particular as a computer program, whereby the blood treatment machine is set up to check the patient's shunt prior to extracorporeal blood treatment, in particular dialysis, according to the principle of fluid bolus delivery to the recirculation at the shunt.

According to the disclosure, the basic idea is realized in that the fluid bolus is formed from the priming fluid which is used in advance of the extracorporeal blood treatment for priming/flushing the extracorporeal blood circuit of the blood treatment machine and is in any case infused into the patient, at least in part, after his venous connection. The recirculation at the shunt is therefore checked immediately after priming/flushing using the priming fluid as a fluid bolus.

In other words, the extracorporeal blood treatment machine according to the disclosure, the detection method according to the disclosure, and the computer program according to the disclosure each provide for using the priming fluid not only for priming/flushing but also for detecting recirculation, and for placing the time of infusion at the end of priming/flushing, that is, before the start of the extracorporeal blood treatment or in a transition period between priming/flushing and the extracorporeal blood treatment.

In this way, the priming/flushing of the extracorporeal blood circuit, which has to be carried out in any case, is used, as disclosed, to detect any recirculation at an early stage, i.e., before or at the start of therapy/extracorporeal blood treatment/extracorporeal dialysis treatment. This ensures that the cannula is correctly positioned on the shunt before or at the start of treatment, making subsequent therapy/extracorporeal blood treatment/extracorporeal dialysis treatment more efficient and safer. In particular, this ensures that the correct cannula position on the shunt does not need to be checked or changed by medical personnel during the course of therapy/extracorporeal blood treatment/extracorporeal dialysis treatment.

An extracorporeal blood treatment machine according to the present disclosure, in particular a dialysis machine, in particular a hemodialysis machine, is intended for extracorporeal blood treatment of a patient's blood and comprises at least:
a dialyzer with a semipermeable membrane;
an extracorporeal blood circulation system with a blood pump that is adapted to convey a fluid, in particular blood, through the dialyzer, wherein the extracorporeal blood circulation extends from an arterial section, which is intended for connection to an arterial shunt section, via a blood inlet of the dialyzer, on a blood side of the semipermeable membrane through the dialyzer, via a blood outlet of the dialyzer and to a venous section which is intended for connection to a venous shunt section;
a dialysis fluid circuit that is adapted to supply fresh dialysis fluid, to convey it through the dialyzer, and to discharge used dialysis fluid, or dialysate, and which extends from a dialysis fluid supply of the blood treatment machine to a dialysis fluid inlet of the dialyzer, on a dialysis fluid side of the semipermeable membrane through the dialyzer, to a dialysate outlet of the dialyzer and to a dialysate sink of the blood treatment machine;
a (first) blood property recording unit adapted to record a blood property in the extracorporeal blood circuit, in particular at the arterial section, and/or a (third) dialysate property recording unit adapted to record a dialysate property at the dialysis fluid outlet; and
a control unit connected to the (first) blood property recording unit and/or the (third) dialysate property recording unit.

According to the disclosure, the blood treatment machine is set up in preparation for extracorporeal blood treatment:
to prime or flush the extracorporeal blood circuit with a priming fluid from the arterial section to the venous section, preferably by controlling the blood pump via the control unit;
to request a connection of the arterial shunt section to the arterial section filled by priming or flushing with priming fluid by an operator, in particular by medical personnel or by the patient himself (in the case of home dialysis), preferably by activating an issuing or display unit of the blood treatment machine via the control unit, and/or to ascertain this connection, in particular on the basis of a sensor signal or an input signal,
to request the connection of the venous shunt section to the venous section filled with priming fluid by flushing, preferably by activating an issuing or display unit of the blood treatment machine via the control unit, and/or to ascertain this connection, in particular on the basis of a sensor signal or an input signal, and
at least depending on the blood property and/or dialysate property recorded after connection of the venous shunt section, to detect and/or quantify and/or check recirculation and/or a recirculation rate at the shunt, preferably by means of the control unit.

According to the disclosure, the blood treatment machine is preferably set up to additionally detect and/or quantify and/or check the recirculation and/or the recirculation rate at the shunt depending on a blood conveying rate or a blood flow and/or a dialysate flow and/or the dialyzer or dialyzer type, preferably by means of the control unit.

According to the disclosure, the extracorporeal blood treatment machine according to a first variant has the (first) blood property recording unit, which is adapted to record the blood property in the extracorporeal blood circuit, in particular at the arterial section, the control unit is signal-connected to the (first) blood property recording unit, and the blood treatment machine, in particular the control unit, is set up to detect and/or quantify and/or check the recirculation and/or the recirculation rate at the shunt depending on the blood property recorded after the connection of the venous shunt section.

According to the disclosure, the extracorporeal blood treatment machine according to a second variant has the (third) dialysate property recording unit, which is adapted to record the dialysate property at the dialysis fluid outlet, the control unit is signal-connected to the (third) dialysate property recording unit, and the blood treatment machine, in particular the control unit, is set up to record and/or quantify and/or check the recirculation and/or the recirculation rate at the shunt depending on the dialysate property recorded after the venous shunt section is connected.

According to the disclosure, the extracorporeal blood treatment machine according to a third variant comprises both the (first) blood property recording unit, which is adapted to record the blood property in the extracorporeal blood circuit, in particular at the arterial section, and the (third) dialysate property recording unit adapted to record the dialysate property at the dialysis fluid outlet, the control unit is signal-connected to both the (first) blood property recording unit and the (third) dialysate property recording unit, and the blood treatment machine, in particular the control unit, is set up to detect and/or quantify and/or check the recirculation and/or the recirculation rate at the shunt depending on the blood property recorded after the connection of the venous shunt section and depending on the dialysate property recorded after the connection of the venous shunt section. The third variant is particularly advantageous, as it allows the measurements obtained on the blood side and the dialysate side to be combined and checked.

According to the disclosure, the blood treatment machine is thus set up to use a priming fluid, which is in any case used for priming/flushing prior to blood treatment or therapy, as a fluid bolus for detecting recirculation. This means that no additional or external bolus administration is necessary. The assessment of the shunt for recirculation, i.e., the assessment of the cannula fit and/or the condition of the shunt, is therefore carried out, as disclosed, before the start or at the start of blood treatment/therapy. This has the advantage that the subsequent blood treatment or therapy for detecting recirculation does not need to be interrupted. Since, as disclosed, recirculation can be detected in advance of blood treatment/therapy and measures can be taken to remedy it, more efficient and safer extracorporeal blood treatment/therapy is possible.

Since the (first) blood property recording unit, preferably arranged on the arterial section of the extracorporeal blood circuit, is in any case usually provided integrated in the machine, there is—with regard to the aforementioned first variant and to the aforementioned third variant—also little technical effort required to carry out the method according to the disclosure, or no conversion or addition to the extracorporeal blood treatment machine is necessary.

According to further development, the control unit is set up to detect recirculation at the shunt at least depending on a deviation of the blood property recorded after connection of the venous shunt section from a reference blood property without recirculation, and/or a value ratio of the blood property recorded after connection of the venous shunt section from a reference blood property without recirculation, and/or an extremum or saddle point of the blood property recorded after connection of the venous shunt section, and/or a gradient of the blood property recorded after connection of the venous shunt section, and preferably to issue a message about the detected recirculation, in particular acoustically and/or visually and/or haptically.

According to further development, the control unit is set up to quantify a recirculation rate at the shunt at least depending on the deviation and/or the value ratio and/or the gradient, and preferably to issue a message on the quantified recirculation rate, in particular acoustically and/or visually and/or haptically.

According to further development, the control unit is set up to determine the deviation as the difference or ratio of the blood properties recorded at different times, or as the difference in area between the blood properties recorded after connecting the venous shunt section and the reference blood properties without recirculation.

According to further development, the control unit is set up to store the recorded blood property at the arterial section (in a storage unit) at the latest when the connection of the venous section is ascertained, in particular its temporal progression.

According to further development, the control unit is set up to store the blood property at least at a first point in time and at a second point in time and to use this to carry out detection and/or quantification.

In the first variant, the arterial shunt section is connected first, then the blood is conveyed using the blood pump or a substitute pump, and only later is the venous shunt section connected. In this case, the first point in time is a point in time at which undiluted blood flows through the blood property recording unit, and the second point in time is a point in time at which, in the event of recirculation occurring, blood diluted with priming fluid flows or would flow through the blood property recording unit.

In a second variant, however, the arterial shunt section and the venous shunt section are connected while the blood pump is stopped. In this case, the first point in time is a point in time at which diluted blood flows through the blood property recording unit, and the second point in time is a point in time at which, in the event of recirculation with priming fluid, less diluted, in particular undiluted, blood flows or would flow through the blood property recording unit.

According to further development, the control unit is set up to receive a recorded or entered positive termination criterion for priming/flushing.

In particular, a second recording unit, at least for recording air or air bubbles, preferably in or on the venous section, and/or an input interface, for example a touch screen, is provided and designed for this purpose.

Preferably, the second recording unit has, in addition to a detector or sensor for recording the air or air bubbles, a detector or sensor for recording the color red or blood.

Preferably, the detectors/sensors for recording the air or air bubbles and the color red or blood are housed in the same sensor housing of the second recording unit.

The preferred positive termination criterion is the absence of bubbles in the extracorporeal blood circuit.

According to further development, the control unit is set up to stop the blood pump upon receipt of the positive termination criterion and to issue a request for connection of the venous shunt section and/or the arterial shunt section, which is directed in particular to the operator.

In the case of the first variant mentioned above, in which the arterial and venous connections are made sequentially, the arterial shunt section is connected first after the positive termination criterion “no air bubbles” has been received. This is followed by blood collection and conveying of blood from the arterial section to the venous section until the color red or blood is recorded by the second recording unit and the blood or substitute pump is stopped by the control unit. You will be prompted to connect the venous shunt section. Once the venous shunt section has been ascertained or entered, blood collection and conveying are continued.

In the second variant mentioned above, once the positive termination criterion “no air bubbles” has been received, the arterial connection and the venous connection are made at the same stop of the blood pump or substitute pump, i.e., in parallel.

According to further development, the control unit is set up to stop the blood pump with a time delay after receiving the positive termination criterion in order to ensure that there are no bubbles in a section of tubing extending downstream of the second recording unit for recording air or air bubbles, and only after the time delay has elapsed, to issue a request to the operator to connect the venous shunt section or the arterial shunt section or the arterial shunt section and the venous shunt section.

According to further development, the control unit is set up to determine the time delay depending on a recorded or determined blood conveying rate and a volume of the tubing section.

Alternatively or in addition, the control unit can be set up with a fixed time delay.

According to further development, the control unit is set up, in particular after receiving the positive termination criterion, to issue a request to connect the venous shunt section and the arterial shunt section during the same interruption of priming/flushing.

According to further development, the control unit is set up to first issue a request to connect the arterial shunt ingate upon receipt of the positive termination criterion and, once connection of the arterial shunt section has been ascertained, to control the conveying of the priming fluid via the venous shunt section into a waste container or line by activating the blood pump, so that the connection of the venous shunt section takes place at a later interruption of the flushing. The waste container or waste conduit may, for example, be designed as a bag or a container or as a waste port or a disposal conduit.

According to further development, the control unit is set up to receive a signal from the second recording unit of the blood treatment machine regarding blood recorded at the venous section, to stop the blood pump, and to issue a request for connection of the venous shunt ingate.

Preferably, the blood treatment machine has an acoustic and/or visual and/or haptic display or issuing unit, in particular a screen, for issuing, in particular for the aforementioned requests and for detecting recirculation and/or quantifying the recirculation rate.

According to the second variant mentioned above or the third variant mentioned above, the extracorporeal blood treatment machine comprises:
a (third) dialysate property recording unit that is connected to the control unit and is set up to record a dialysate property at the dialysis fluid outlet. According to the disclosure, the control unit is set up according to these variants to detect and/or quantify and/or check the recirculation and/or the recirculation rate depending on the dialysate property recorded after the connection of the venous shunt section.

The detection of recirculation and/or the quantification of the recirculation rate can thus be performed redundantly. On the one hand, by means of the blood-side, first blood property recording unit, and on the other hand, by means of the dialysate-side, third dialysate property recording unit.

Since, according to the disclosure, detection and/or quantification can apparently be performed not only unilaterally on the basis of the recorded blood property or the recorded dialysate property, but bilaterally, i.e., on the basis of both the recorded blood property and the recorded dialysate property, it is possible to reliably check the position of the cannulas on the shunt and/or the condition of the shunt by means of both recorded properties.

Preferably, the control unit is set up to form an average value from the recirculation rates quantified on the blood side and on the dialysate side in order to increase the measurement accuracy and/or reliability of the information regarding any recirculation that may be present. The averaging can be done, for example, by weighting the two recirculation rates differently.

Preferably, the control unit is set up to check the recirculation rates quantified on the blood side and dialysate side for plausibility. If both recirculation rates are approximately the same, the determined recirculation rates can be trusted. If they differ, the control unit is preferably set up to issue a message to this effect.

According to a further development, the control unit is set up to detect and/or quantify and/or check the recirculation at the shunt and/or the recirculation rate at the shunt depending on a deviation of the dialysate property recorded after connecting the venous shunt section from a reference dialysate property without recirculation, and/or a value ratio of the dialysate property recorded after connecting the venous shunt section to a reference dialysate property without recirculation, and/or depending on a gradient of the dialysate property recorded after connecting the venous shunt section.

According to the disclosure, the blood treatment machine described above is set up to perform each of the steps of the computer-implemented detection method described below. If one of the steps is to be/must be performed manually, for example by the operator, in particular by medical operating personnel or the patient themselves—for example, connecting the shunt—the blood treatment machine is preferably set up to issue an acoustic and/or visual and/or haptic request or message that prompts the operator to perform the manual step.

Preferably, the control unit of the blood treatment machine has a memory unit in which the detection method according to at least one aspect of the following disclosure and/or a computer program according to at least one aspect of the following disclosure is computer-implemented or stored for execution.

The blood treatment machine is preferably set up to perform this detection method in preparation for extracorporeal blood treatment and to terminate it before or at the start of extracorporeal blood treatment or therapy.

A computer-implemented detection method is provided according to the present disclosure for detecting/ascertaining recirculation at a shunt of an extracorporeal blood circuit of a blood treatment machine in preparation for extracorporeal blood treatment. The blood treatment machine has a dialyzer with a semipermeable membrane, at which the blood conveyed in the extracorporeal blood circuit can be brought into material exchange with a dialysis fluid of a dialysis fluid circuit. According to the disclosure, the method comprises the following steps:
priming/flushing (hereinafter referred to as flushing) of the extracorporeal blood circuit with a priming fluid, from an arterial section of the extracorporeal blood circuit, which is intended for connection to an arterial shunt section, to a venous section of the extracorporeal blood circuit, which is intended for connection to a venous shunt section. Priming/flushing can be performed with the aid of gravity and/or by means of a blood pump or substitute pump of the blood treatment machine;
(requesting and/or ascertaining) connection of the arterial shunt section to the arterial section of the extracorporeal blood circuit filled by priming or flushing with priming fluid;
(requesting and/or ascertaining) connection of the venous shunt section to the venous section of the extracorporeal blood circuit filled by priming or flushing with priming fluid;
recording a blood property in the extracorporeal blood circuit, preferably at the arterial section of the extracorporeal blood circuit, by means of a (first) blood property recording unit of the blood treatment machine and/or recording a dialysate property at a dialysis fluid outlet of the dialyzer by means of a (third) dialysate property recording unit of the blood treatment machine; and
detecting and/or quantifying and/or checking any recirculation present at the shunt, at least depending on the blood property and/or dialysate property recorded after connecting the venous shunt section, preferably by means of a control unit of the blood treatment machine connected to the (first) blood property recording unit and/or the (third) dialysate property recording unit.

Accordingly, the priming fluid used for rinsing prior to blood treatment or therapy is used as a bolus for detecting recirculation. This means that no additional or external bolus administration is necessary. The assessment of the shunt for recirculation, i.e., the assessment of the cannula position and/or the condition of the shunt, is therefore carried out, as disclosed, before or at the start of blood treatment/therapy. This has the advantage that the subsequent blood treatment/therapy for detecting recirculation no longer needs to be interrupted. Since, as disclosed, recirculation can be detected in advance of or at the start of blood treatment/therapy and measures can be taken to remedy the situation, more efficient and safer extracorporeal blood treatment or therapy is possible.

Since the (first) blood property recording unit is usually integrated into the machine at the arterial section of the extracorporeal blood circuit anyway, the technical effort required to carry out the method disclosed is low for some method variants, or no conversion or addition to the extracorporeal blood treatment machine is necessary.

The blood property recorded can be chemical, such as a concentration, or physical, such as absorption. The prerequisite for successful detection of recirculation is that the blood property intended for recording changes when the blood is diluted with priming fluid and that this change can be recorded so that it can be used as a measure of blood dilution or recirculation.

The blood property recorded is, in particular, the concentration of a substance in the blood. This includes, for example, hemoglobin concentration or any other concentration that can be recorded by sensory means and that changes when diluted with the priming fluid. Other possible blood properties include relative blood volume or hematocrit value. Alternatively or in addition, the speed of sound can be recorded as a blood property, as blood and priming fluid differ in terms of their speed of sound.

The first blood property recording unit is specially adapted to the blood property to be recorded.

The priming fluid is preferably an isotonic NaCl solution, in particular 0.9% or 9 g NaCl per 1000 ml, or fresh dialysis fluid.

The respective signal connection can be wired or wireless, for example via Bluetooth.

According to further development, recirculation at the shunt is detected at least depending on a deviation of the blood property recorded after connecting the venous shunt section from a reference blood property without recirculation, and/or a value ratio of the blood property recorded after connecting the venous shunt section to a reference blood property without recirculation, and/or depending on an extremum or saddle point of the blood property recorded after connecting the venous shunt section, and/or depending on a gradient of the blood property recorded after connecting the venous shunt section.

According to further development, one step involves quantifying a recirculation rate at the shunt depending on the deviation and/or the value ratio and/or the gradient using the control unit.

According to further development, the deviation is determined as the difference or ratio of the blood property recorded at different times or as the difference in area between the blood property recorded after connecting the venous shunt section and the reference blood property without recirculation.

According to further development, the step of recording the blood properties at the arterial section is performed at the latest when the venous shunt section is connected to the venous section.

According to further development, the step of recording the blood properties at the arterial section is performed at least at a first point in time and at a later second point in time. The first point in time is preferably a point in time at which undiluted blood flows through the blood property recording unit, and the second point in time is a point in time at which, in the event of recirculation occurring, blood diluted with priming fluid flows or would flow through the blood property recording unit.

According to further development, the step of connecting the arterial shunt section takes place after a step of recording or entering a positive termination criterion for flushing, in particular the absence of bubbles, via a (second) recording unit or input unit of the blood treatment machine. Preferably, the (second) recording unit is designed to record air or air bubbles in the venous section.

According to one variant, the steps of connecting the venous shunt section and connecting the arterial shunt section are performed during the same interruption of flushing, in particular during the same stop of the blood pump.

According to another variant, between the step of connecting the arterial shunt ingate and the step of connecting the venous shunt section, there is a step of conveying the priming fluid via the venous section into a waste container, in particular via the blood pump. In this way, the steps of connecting the venous shunt section and connecting the arterial shunt section are performed with different interruptions of the flushing, or with different stops of the blood pump, and the amount of priming fluid infused into the patient is minimized.

According to further development, the step of connecting the venous shunt section takes place after a step of recording blood at the venous section, via a second recording unit of the blood treatment machine and, if blood has been recorded, after a step of stopping the blood pump.

According to the disclosure, alternatively or additionally, a step of recording a dialysate property at a dialysis fluid outlet of the dialyzer by means of a (third) dialysate property recording unit of the blood treatment machine is provided. In the case of a combination, the recorded blood property and the recorded dialysate property can be used to check the cannula position or the condition of the shunt by detecting recirculation.

A mean recirculation rate can be determined from both properties in order to increase the accuracy of the recirculation rate quantification.

The blood-side recirculation rate and the dialysate-side recirculation rate can be weighted in the calculation of the mean value or the averaging. Alternative methods of averaging are possible.

In addition, the recorded dialysate property can be used to check the plausibility of the recirculation rate quantified using the recorded blood property. If the recirculation rates quantified on the basis of the recorded blood properties and the recorded dialysate properties are approximately the same, the determined recirculation rate can be trusted.

If they differ, one of the quantified recirculation rates appears to be incorrect, in which case the method includes a step to issue a message indicating that the recirculation rates are inconsistent and/or that the shunt should be checked.

According to further development, the step of detecting recirculation at the shunt and/or quantifying the recirculation rate at the shunt is therefore performed additionally, depending on the dialysate properties recorded after connecting the venous shunt section, using the control unit.

According to further development, a step is provided for checking the detected recirculation and/or the quantified recirculation rate depending on the dialysate properties recorded after connecting the venous shunt section.

According to further development, the step of detecting recirculation at the shunt and/or quantifying the recirculation rate at the shunt and/or checking it depending on a deviation of the dialysate property recorded after connecting the venous shunt section from a reference dialysate property without recirculation, and/or checking it depending on a gradient of the dialysate property recorded after connecting the venous shunt section.

A computer program according to the present disclosure comprises instructions that, when executed by a computer, cause the computer to perform the method steps of the detection method according to at least one aspect of the foregoing description.

### BRIEF DESCRIPTION OF THE DRAWINGS

The disclosure will be explained in more detail below by means of preferred embodiments, with the aid of figures.

FIG. 1 shows a fluidic circuit diagram of an extracorporeal blood treatment machine according to a preferred embodiment;

FIG. 2 shows a temporal progression of a blood property at the shunt, once with and once without recirculation, when performing a detection method according to a preferred embodiment;

FIG. 3 shows a temporal progression of a blood property at the shunt, twice with and once without recirculation, when performing a detection method according to a further embodiment;

FIG. 4 shows a temporal progression of a dialysate property, twice with recirculation and once without recirculation, when performing a detection method according to a further embodiment;

and

FIG. 5 shows a flowchart of a computer-implemented control method according to a preferred embodiment.

The figures are schematic in nature and are intended only to aid understanding of the disclosure. Identical elements are marked with the same reference signs. The features of the various embodiments can be interchanged.

### DETAILED DESCRIPTION

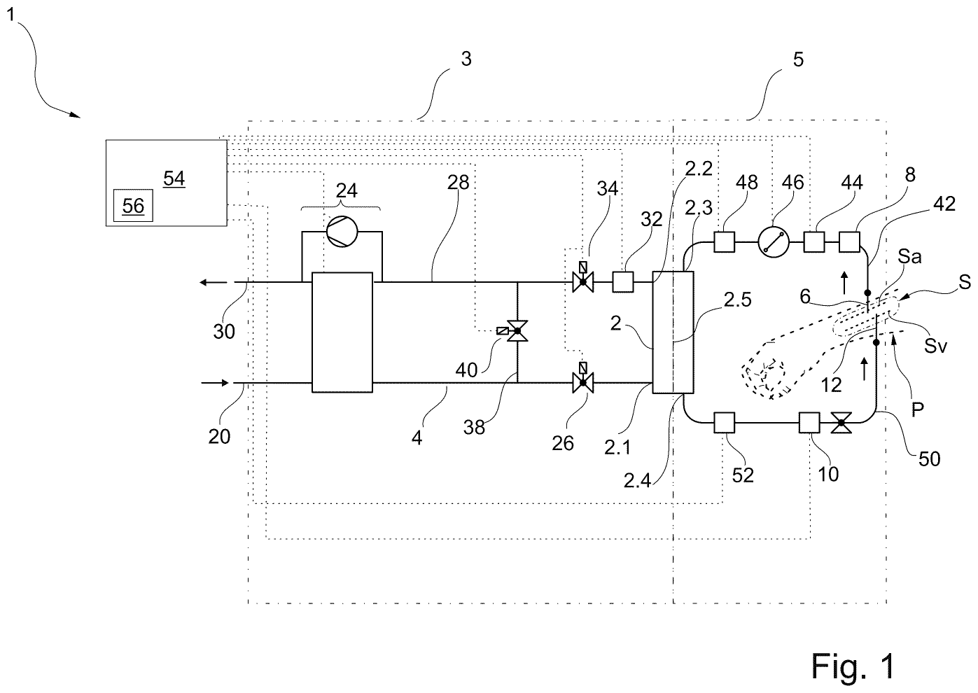

FIG. 1 shows a schematic view of a fluidic circuit diagram of an extracorporeal blood treatment machine 1 designed as a hemodialysis machine for extracorporeal blood treatment of blood from a patient P according to a preferred embodiment of the present disclosure.

The extracorporeal blood treatment machine 1 has a dialyzer 2 with a dialysis fluid circuit 3 and an extracorporeal blood circuit 5 as its central component. The dialyzer 2 has a dialysis fluid inlet 2.1 and a dialysate outlet 2.2 on the dialysis fluid side and a blood inlet 2.3 and a blood outlet 2.4 on the blood side. Inside, the dialyzer 2 is separated into a dialysis fluid side and a blood side by hollow fibers of a semipermeable membrane 2.5.

The dialysis fluid inlet 2.1 can be fluidically connected, and is in particular connected, to a dialysis fluid supply 20 via a dialysis fluid inflow 4 and a balancing device 24. At the dialysis fluid supply 20, fresh dialysis fluid is supplied from a permeate, a basic concentrate, and an acidic concentrate. A valve 26 is arranged in the dialysis fluid inflow 4 to shut off the dialysis fluid inlet 2.1.

The dialysate outlet 2.2 can be fluidically connected, and is in particular connected, via a dialysate drain 28 and the balancing device 24 to a disposal outlet 30 for used dialysis fluid/dialysate. The balancing device 24 ensures that a desired volume of excess water can be removed from the patient's blood during ultrafiltration.

In the dialysate drain 28, between the dialysate outlet 2.2 and the disposal outlet 30, the following are arranged in series in terms of fluid flow: a third recording unit 32 for recording a dialysate property D at the dialysate outlet, in particular for recording a component in the used dialysate, and an actuable valve 34, preferably of the same design as valve 26, for shutting off dialysate outlet 2.2.

The third recording unit 32 can be designed as an optical measuring device with a radiation emitter in the form of an LED and a photodetector, or it can be designed, for example, as a conductivity measuring device, in particular a temperature-compensated conductivity measuring device.

Alternatively or in addition, further recording units may be provided at dialysate outlet 2.2 in order to record further components in the spent dialysate. For example, one of the recording units could record conductivity with reference to a component A in the dialysate, and another of the recording units could record extinction with reference to a component B in the dialysate.

The dialysis fluid inflow 4 can be fluidically connected to the dialysate drain 28 via a bypass flow path 38. An actuable valve 40, preferably of the same design as valves 26, 34, is arranged in bypass flow path 38.

With the help of valves 26, 34, and 40, the dialysis fluid circuit 3 can be switched via a control unit 54 of the blood treatment machine 1, as disclosed, into a main circuit in which dialysis fluid is supplied via the dialysis fluid inflow 4 at the dialysis fluid inlet 2.1 and is conveyed through the dialyzer 2 to the dialysate outlet 2.2.

In the main shut-off circuit, valves 26 and 34 of the dialysis fluid inflow 4 and the dialysate drain 28 are actuated to their open position via the control unit 54, while valve 40 in the bypass flow path 38 is actuated to its closed position so that the bypass flow path 38 is blocked.

Furthermore, the dialysis fluid circuit 3 can be switched into a bypass circuit by means of the fluidic switching means/valves 26, 34, and 40, in which the dialysis fluid inflow 4 is fluidically separated from the dialysis fluid inlet 2.1 and the dialysate drain 28 is fluidically separated from the dialysate outlet 2.2, while the dialysis fluid inflow 4 is fluidically connected to the dialysate drain 28 via the bypass flow path 38. This prevents the fresh dialysis fluid from flowing through the dialyzer 2, but instead flows from the dialysis fluid inflow 4—bypassing the dialyzer 2—via the open bypass flow path 38 directly to the dialysate drain 28. In the bypass circuit, the switching of valves 26, 34, 40 is reversed relative to the main circuit, i.e., valves 26 and 34 of the dialysis fluid inflow 4 and the dialysate drain 28 are actuated to their closed position via the control unit 54, while valve 40 in the bypass flow path 38 is actuated to its open position.

The extracorporeal blood circuit 5 has an arterial (tubing) section 42, which is intended for connection to an arterial shunt section Sa of the shunt S. The connection is preferably made by means of an arterial cannula 6 that punctures the arterial shunt section Sa. The extracorporeal blood circuit 5 further has a venous (tubing) section 50, which is intended for connection to a venous shunt section Sv of the shunt S. The latter connection is preferably made by means of a venous cannula 12 puncturing the venous shunt section Sv.

In the extracorporeal blood circuit 5, a first recording unit 8 for recording a blood property B, an arterial pressure sensor 44, a blood pump 46, and a blood inlet pressure sensor 48 are arranged between the arterial section 42 and the blood inlet 2.3 of the dialyzer 2. A blood outlet pressure sensor 52, a second recording unit 10 for detecting blood and air or air bubbles, and a shut-off valve are arranged between the blood outlet 2.4 of the dialyzer 2 and the venous section 50.

During normal extracorporeal blood treatment, blood is withdrawn from the patient via the arterial cannula 6 and fed to the dialyzer 2 via the blood inlet 2.3. The blood is passed through the dialyzer 2 in countercurrent to the dialysis fluid of the dialysis fluid circuit 3, where it is freed of urinary components and excess water and then returned/fed back to the patient P in a purified state. For this purpose, blood is taken from patient P at blood outlet 2.4 and fed via the venous section 50 and the venous cannula 12 to the venous section Sv of the shunt S.

The control unit 54 is connected to valves 26, 34, and 40 via signals so that it can control/activate them in the main circuit and bypass circuit described above. In addition, the control unit 54 is connected to the pressure recording units 44, 48, 52, to the first, second and third recording units 8, 10, 32, to the balancing device 24 and to the blood pump 46.

The following is a description of a computer-implemented detection method according to the disclosure for the blood treatment machine 1 shown in FIG. 1, with reference to FIGS. 2, 3, 4, and 5.

In brief, the detection method disclosed herein provides for assessing the position of the cannulas 6, 12 on the shunt S and the condition of the shunt S by means of a pre-dialysis qualitative detection of recirculation r and/or a quantitative determination of a recirculation rate R.

FIG. 5 shows a flow chart of the computer-implemented detection method for detecting and quantifying recirculation r and recirculation rate R present at shunt S according to a preferred embodiment, in which as little priming fluid as possible is infused into the patient. The detection method according to the disclosure comprises the following steps:

starting S0 of the detection method, in particular by means of an input by a user at a user interface of the blood treatment machine 1 or via the control unit 54 according to FIG. 1.

priming/flushing S1 of the extracorporeal blood circuit 5 according to FIG. 1, starting from its arterial section 42, which is intended for connection to the arterial shunt section Sa, to its venous section 50, which is intended for connection to the venous shunt section Sv, with priming fluid. priming/flushing S1 is preferably performed by activating the blood pump 46 according to FIG. 1.

This is followed by a step of recording S2 a positive termination criterion for priming/flushing, preferably at least the absence of bubbles in the extracorporeal blood circuit 5 recorded by the second recording unit 10.

Based on the recorded termination criterion, the blood pump is stopped and step of connecting S3 the arterial shunt section Sa to the arterial section 42 filled by priming/flushing with priming fluid is performed. The venous shunt section Sv should preferably remain disconnected.

The blood pump 46 performs a step of conveying S4 the priming fluid via the venous section 50 into a waste container (not shown in FIG. 1) or into the dialysate drain 28, so that the priming fluid can be discarded via the disposal outlet 30. Due to the arterial connection, blood is simultaneously withdrawn from the arterial shunt section Sa and conveyed past the first recording unit 8. In this way, the priming fluid is displaced from the extracorporeal blood circuit 5 into the waste container and the amount of priming fluid to be infused later into the patient for detection of recirculation is minimized. Secondly, the extracorporeal blood circuit 5 is gradually filled with blood.

When the blood reaches the venous section 50 according to FIG. 1, a step of recording S5 the blood at the venous section 50 via the second recording unit 10 follows and, if or as soon as blood has been/is recorded, a step of stopping S6 the blood pump 46 takes place.

With the blood pump 46 stopped, a step can now be taken to connect S7 the venous shunt section Sv to the venous section 50 filled by priming/flushing with priming fluid, and the blood pump 46 can be restarted.

In an alternative embodiment of the method, the steps of connecting S3 the arterial shunt section Sa to the arterial section 42 filled by priming/flushing with priming fluid and connecting S7 the venous shunt section Sv to the venous section 50 filled by priming are performed at the same stop of the blood pump 46 (after the step of recording S2 the positive termination criterion for priming/flushing). This means that a priming fluid bolus infused into patient P is larger than in the embodiment of the method according to FIG. 5.

At this point in time at the latest, i.e., when both shunt sections Sa and Sv are connected and blood pump 46 is back in operation, step of recording S8 blood property B at the arterial section 42 is performed by means of the first recording unit 8.

The step of recording S8 of the blood property B is performed at least twice: at an initial point in time t1, at which the priming fluid is displaced from the arterial section 42 by blood and the first recording unit 8 records the blood property B in an undiluted state, i.e., at which undiluted blood flows through the first recording unit 8, and at a later, second point in time t2, at which, in the event of recirculation, blood diluted with priming fluid flows or would flow through the first recording unit 8, in particular if the priming fluid conveyed/displaced into the venous shunt section Sv recirculates and mixes with blood in the arterial section 42. The diluted blood is conveyed by the blood pump 46 from the arterial shunt section Sa into the arterial section 42 and passes through the first recording unit 8, where a time curve Br, Br′, Br″ corresponding to the dilution of the blood property B is recorded and reported to the control unit 54. The recorded history Br, Br′, Br″ of the blood property B is stored in the memory unit 56 of the control unit 54 for evaluation, in particular for the detection of recirculation.

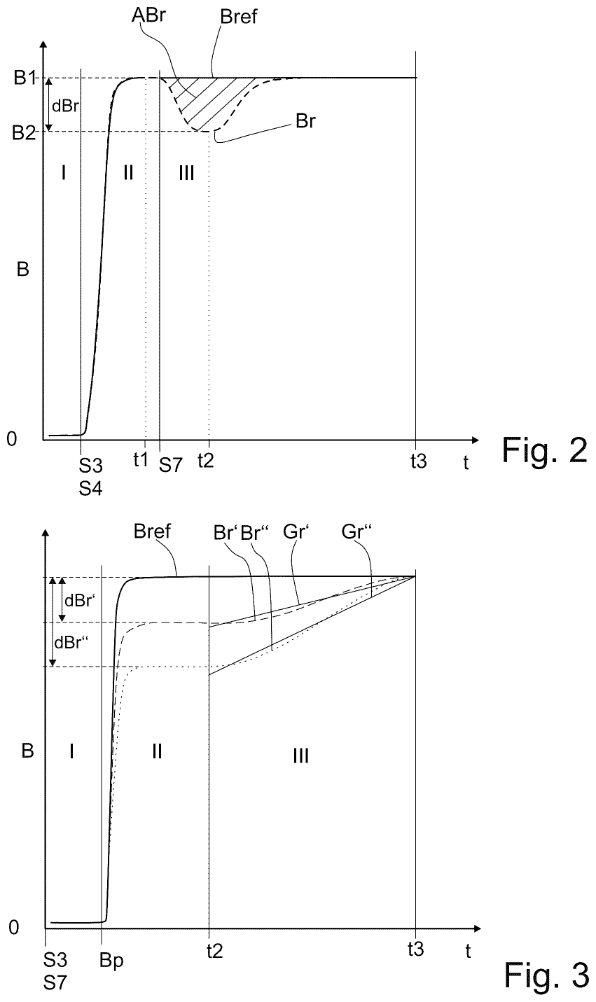

A detection step S9 is performed to detect recirculation r at the shunt S depending on the blood properties Br, Br′, Br″ recorded after connecting S7 the venous shunt section Sv, using the control unit 54. Recirculation r at the shunt S is detected by the control unit 54 by analyzing the time curve Br, Br′, Br″ of the blood property B after connecting S7 of the venous shunt section Sv. A deviation from a reference blood property without recirculation Bref can be used as a measure of recirculation r. The deviation can be determined, for example, as the difference in area ABr (see FIG. 2) or as the value difference dBr, dBr′, dBr″ (see FIGS. 2 and 3) or as the value ratio of Br, Br′, Br″ to Bref of the blood property Br, Br′, Br″ recorded at different times t1, t2. An alternative or supplementary option is to determine an extremum (see FIG. 2) or a saddle point with a subsequent gradient Gr′, Gr″ (see FIG. 3) of the recorded blood property Br, Br′, Br″ and use this as a measure.

This is followed by a step of quantifying S10 of the recirculation rate R at the shunt S at least depending on the deviation ABr, dBr, dBr′, dBr″ and/or the gradient Gr′, Gr″ and/or the value ratio of Br, Br′, Br″ to Bref, by means of the control unit 54. In addition, step of quantifying S10 the recirculation rate R at the shunt S can be performed depending on further parameters, in particular depending on the blood conveying rate or the blood flow, the dialysate flow, etc.

Alternatively or in addition to steps S8, S9, and S10, a step of recording S11 a dialysate property D at the dialysis fluid outlet 2.2 of the dialyzer 2 is performed by means of the third recording unit 32 of the blood treatment machine 1 (see FIG. 1).

Depending on the dialysate properties Dr, Dr′ recorded after connecting S7 of the venous shunt section Sv, a step of checking S12 the detected recirculation r and/or the quantified recirculation rate R is preferably performed.

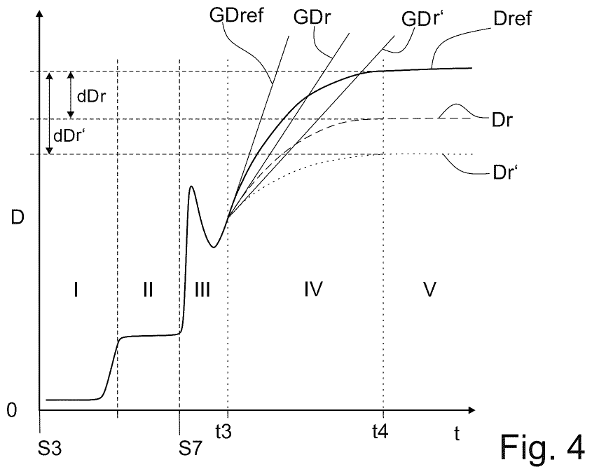

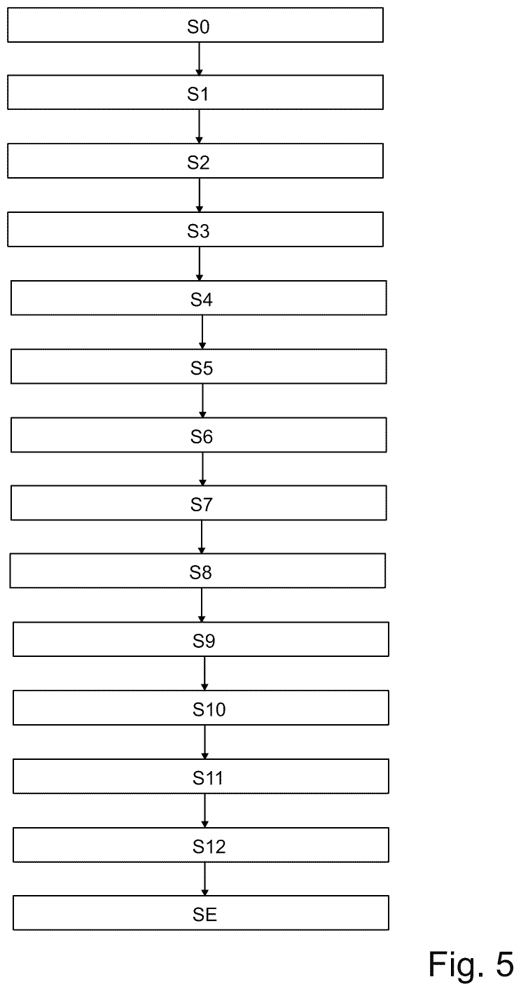

Preferably, the step of detecting S9 the recirculation r and/or quantifying S10 the recirculation rate R and/or checking S11 them is performed depending on a deviation dDr, dDr′ of the dialysate property Dr recorded after connecting S7 the venous shunt section Sv, Dr′ of a reference dialysate property without recirculation Dref and/or a value ratio of Dr, Dr′ to Dref and/or a gradient GDr, GDr′ of the dialysate property Dr, Dr′ recorded after connecting S7 the venous shunt section Sv (see FIG. 4) and, if necessary, depending on further parameters, such as in particular depending on the blood conveying rate or the blood flow, the dialysate flow, etc.

Depending on the detected recirculation r or quantified recirculation rate R, measures to eliminate recirculation at the shunt S are taken before starting blood treatment or therapy.

This is followed by the step of ending SE of the detection method.

The computer-implemented detection method described above is completed with the step of ending SE, essentially before the extracorporeal blood treatment or therapy begins on patient P using the extracorporeal blood treatment machine 1.

The start of extracorporeal blood treatment or therapy on patient P is preferably marked by the start of ultrafiltration. Only then is excess water removed from the blood in addition to substances that need to be excreted in urine.

FIG. 2 shows the course of blood property Br with recirculation present in response to the bolus administration described above after step of connecting S7 the venous shunt section Sv. A reference curve of the blood property, or a reference blood property Bref, is also shown, which results when there is no recirculation at the shunt S.

In phase I according to FIG. 2, the arterial shunt section Sv is connected (see step S3) and the blood pump 46 conveys blood (see step S4). Accordingly, the blood of patient P displaces the priming fluid located in the arterial section 42 and passes through the first recording unit 8 according to FIG. 1. Accordingly, the signal of blood property B of the first recording unit 8 rises sharply in phase II according to FIG. 2 until it reaches its maximum B1 at approximately point in time t1.

Since the venous shunt section Sv is not yet connected at this point in time t1 (see step S7 not yet performed) and, as described above, the priming fluid is being conveyed from the venous section 50 into the above-mentioned waste container, no recirculation can be determined/detected here yet.

As soon as blood 10 is recorded at the second recording unit (see step S5), the blood pump 46 is stopped via the control unit 54 and the venous shunt section Sv is connected (see step S7). The blood pump 46 is then restarted.

If recirculation occurs, the arterial blood at the arterial shunt section Sa is diluted by the priming fluid flowing in from the venous section 50 and recirculating. The diluted blood passes through the first recording unit 8, which records a decrease in the recorded blood property Br corresponding to the dilution/recirculation and reports this to the control unit 54.

Once the bolus of priming fluid in the venous section 50 has been displaced, this dilution decreases again and in phase III, the recorded blood property Br rises again. Here, a minimum B2 of blood property B is reached at approximately point in time t2.

Detection and/or quantification of recirculation r/recirculation rate R can now be performed, for example, depending on the difference in area ABr and/or the two values B1, B2 (see steps S9, S10).

For example, the difference in area ABr, which is bounded by the points in time t1, t2, the reference blood property Bref, and the blood property Br recorded after connecting the venous shunt section Sv, is proportional to the recirculation rate R.

On the other hand, the recirculation rate can be calculated using the two values B1 and B2 using the following equation:

$$R ( \% ) = ( 1 - B 2 / B 1 ) * 100 \%$$

If the quantified recirculation rate R exceeds a predetermined limit stored in the control unit 54, the control unit 54 can trigger an acoustic and/or visual and/or haptic signal to be issued by a display unit of the blood treatment machine 1. This may include, for example, instructing operating personnel to check the shunt S and/or the correct position of cannulas 6 and 12.

If there is no recirculation, the first recording unit 8 records a blood property B curve that corresponds to that of the reference blood property Bref shown in FIG. 2. After connecting the venous shunt section Sv (see step S7), the recorded blood property Br therefore remains constant (like Bref) for the time being.

FIG. 3 shows an embodiment of the detection method that differs from the embodiment described above. Essentially, the difference lies in the fact that the connection of the arterial shunt section Sa (see step S3) and the venous shunt section Sv (see step S7) takes place simultaneously, i.e., during the same stop of the blood pump 46.

The priming fluid no longer needs to be conveyed into the pre-described waste container, which means that all of the priming fluid in the extracorporeal blood circuit 5 is infused.

FIG. 3 shows the time curves of the reference blood property Bref (0%), the blood property Br′ (15%) and the blood property Br″ (30%) for three different recirculation rates R of 0%, 15%, and 30%.

In phase I, as shown in FIG. 3, patient P is treated without bloodletting. This means that the shunt S of patient P is punctured with both the arterial cannula 6 and the venous cannula 12, or the arterial cannula 6 is connected to the arterial section 42 and the venous cannula 12 is connected to the venous section 50 (see steps S3 and S7). At this point in time, the extracorporeal blood circuit 5 is completely filled with priming fluid and free of bubbles, as described above. In phase II, blood pump 46 starts (point in time Bp). Arterial blood is thus withdrawn from the patient and venous priming fluid is infused at the same time. The signal at the first recording unit 8 rises as a result and reaches a saddle point in phase II, from which point the signal remains constant for approximately 1 minute, depending on the blood flow rate. The solid line shows the course of the reference blood property Bref, i.e., the case where there is no recirculation or the recirculation rate is 0%. The dashed line shows the progression of the blood property Br′ at an average recirculation rate R of approximately 15%, and the dotted line shows a higher recirculation rate of approximately 30%.

Depending on the recirculation rate R, the level of the saddle point in phase II varies, as the venously infused priming fluid mixes with the blood withdrawn from the arteries. In phase III, it can be seen how the priming fluid is increasingly displaced from the extracorporeal blood circuit 5 and replaced by blood originating from the patient P. This causes the signal at the first recording unit 8 to rise and reach a maximum value of Bref without recirculation.

A respective gradient Gr′, Gr″ in phase III, in particular an average gradient, can be used as a measure for the recirculation rate R.

In addition to the course of the blood property Br, Br′, Br″ in phase III and/or the gradient Gr′, Gr″ in phase III and/or the difference in area ABr in phase III, and/or the difference dBr, dBr′, dBr″ can be used to quantify the recirculation rate R—regardless of the embodiment—the blood flow and/or the dialysis fluid flow and/or the ultrafiltration rate and/or a measured value at the transition from phase II to phase III, and/or the dialyzer 2 used.

Substances in the blood can pass through the semipermeable membrane 2.5 from the blood into the dialysis fluid by diffusion. Therefore, in addition or as an alternative—and analogous to the detection/quantification based on the recorded blood property B—a signal response to the aforementioned bolus administration can be recorded and evaluated on the dialysate side using priming fluid. The data is recorded by the third recording unit 32 and evaluated by the control unit 54. The third recording unit 32 may, for example, be an optical sensor that records an absorption property of the dialysate at the at least one wavelength. In this case, all substances that absorb light at the at least one wavelength would contribute to absorption.

FIG. 4 now shows, by way of example and schematically, the signal curve Dr, Dr′ of the dialysate property D recorded at the third recording unit 32, as it results for different recirculation rates R. In phase I, patient P is set up. This means that patient P is punctured with arterial cannula 6 or that arterial cannula 6 is connected to arterial section 42 (see step S3). The blood pump 46 is started and continues to convey until blood is detected at the venous section 50 by the second recording unit 10, which is designed as a blood and air detector. If the blood treatment machine 1 is in the above-mentioned main circuit, i.e., dialysis fluid is flowing through the dialyzer 2, substances from the blood will reach the dialysate side. This leads to a signal increase at the third recording unit 32, which can be observed towards the end of phase I. If blood treatment machine 1 were instead in bypass, no signal change would be measurable in this phase. The duration of phase I depends on the blood flow setting and the filling volume of the extracorporeal blood circuit 5 including the dialyzer 2 and can range from a few seconds to approximately 2.5 minutes. In phase II, the blood pump 46 is not running. Patient P is now also connected to the venous line (see step S7). However, even when the blood pump 46 is not running, a substance exchange takes place at the semipermeable membrane 2.5, wherein substances that need to be excreted in urine, including light-absorbing substances, pass through the membrane 2.5 and reach the dialysis fluid side. Typically, when the blood pump 46 is stopped, the blood treatment machine 1 is located in the above-mentioned bypass circuit. This prevents dialysis fluid or dialysate from flowing along the third recording unit 32, so that the signal there remains constant in accordance with FIG. 4. The bypass circuit in phase II is not mandatory, which means that the signal in phase II could also drop again. In the fastest case, phase II lasts only a few seconds. In phase III, the blood pump 46 is restarted. At the latest in phase III, blood treatment machine 1 is located in the above-mentioned main circuit so that relevant signal curves can be recorded at the third recording unit 32. If blood treatment machine 1 was previously in phase II in the bypass circuit, a saturation process took place in dialyzer 2 on the dialysis fluid side. Substances that are small enough to pass through the membrane 2.5 diffuse to the dialysis fluid side of the dialyzer 2. This “saturation bolus” is noticeable as a brief signal increase in phase III.

The blood flow can be preset or changed (increased). Phase III is completed in less than a minute.

Investigations have shown that the signal of the recorded dialysate property Dr, Dr′ varies depending on the recirculation rate R from phase IV onwards. The solid line shows the course of the recorded dialysate property D without recirculation, i.e., the reference dialysate property Dref at R=0%. The dashed line shows the course of the recorded dialysate property Dr at medium recirculation (e.g., R=15%) and the dotted line shows the course of the recorded dialysate property Dr′ at increased recirculation (e.g., R=30%). It is evident that the gradient GD of the recorded dialysate property D decreases with increasing recirculation rate R. Phase IV is usually completed after approximately 30 seconds. The signal has stabilized in phase V and would decrease during further blood treatment or dialysis therapy, as more substances excreted in the urine would be removed.

In addition to the signal behavior in phase IV, the blood flow, the dialysate flow, the measured value at the transition from phase IV to phase V, and other parameters such as the dialyzer 2 used can be used in combination to determine the recirculation rate R.

### LIST OF REFERENCE SIGNS

1 extracorporeal blood treatment machine
2 dialyzer
2.1 dialysis fluid inlet
2.2 dialysate outlet
2.3 blood inlet
2.4 blood outlet
2.5 semipermeable membrane
3 dialysis fluid circuit
4 dialysis fluid inflow
5 extracorporeal blood circulation
6 arterial cannula
8 first recording unit
10 second recording unit
12 venous cannula
20 dialysis fluid supply
24 balancing device
26 first valve
28 dialysate drain
30 disposal outlet
32 third recording unit
34 second valve
38 bypass flow path
40 third valve
42 arterial (tubing) section
44 arterial pressure sensor
46 blood pump
48 blood inlet pressure sensor
50 venous (tubing) section
52 blood outlet pressure sensor
54 control unit
56 memory unit
S0 step: starting the detection method
S1 step: flushing the extracorporeal blood circuit
S2 step: recording the flush termination criterion
S3 step: connecting the arterial shunt section
S4 step: conveying the priming fluid
S5 step: recording blood from the venous section
S6 step: stopping the blood pump
S7 step: connecting the venous shunt section
S8 step: recording the blood property
S9 step: detecting recirculation
S10 step: quantifying the recirculation rate
S11 step: recording the dialysate property
S12 step: checking recirculation or recirculation rate
SE step: ending the detection method
P patient
S shunt
Sa arterial shunt section
Sv venous shunt section
B blood property
Bref reference blood property
Br, Br′, Br″ blood property according to venous connection
dBr, dBr′, dBr″ blood property difference
ABr blood property difference in area
D dialysate property
Dref reference dialysate property
Dr, Dr′ dialysate property after venous connection
dDr, dDr′ dialysate property difference
GDref reference gradient dialysate property
GDr, GDr′ gradient dialysate property

1. An extracorporeal blood treatment machine for extracorporeal blood treatment of blood of a patient, the extracorporeal blood treatment machine comprising:
a dialyzer with a semipermeable membrane;
a shunt having an arterial shunt section and a venous shunt section;
an extracorporeal blood circuit with a blood pump adapted to convey a fluid through the dialyzer, the extracorporeal blood circuit extending from an arterial section, which is supplied for connecting to an arterial section, via a blood inlet of the dialyzer, on a blood side of the semipermeable membrane through the dialyzer, via a blood outlet of the dialyzer and to a venous section, the venous section configured for connecting the venous shunt section;
a dialysis fluid circuit which is adapted to supply fresh dialysis fluid, to convey it through the dialyzer and to discharge used dialysis fluid or dialysate, and which extends from a dialysis fluid supply of the extracorporeal blood treatment machine to a dialysis fluid inlet of the dialyzer, on a dialysis fluid side of the semipermeable membrane through the dialyzer, to a dialysate outlet of the dialyzer and to a dialysate sink of the extracorporeal blood treatment machine;
a blood property recording unit, which is adapted to record a blood property in the extracorporeal blood circuit, and/or a dialysate property recording unit, which is adapted to record a dialysate property at the dialysate outlet; and
a control unit connected to the blood property recording unit and/or the dialysate property recording unit,
the extracorporeal blood treatment machine being configured, in preparation for extracorporeal blood treatment, to:
prime or flush the extracorporeal blood circuit with a priming fluid from the arterial section to the venous section,
request a connection of the arterial shunt section to the arterial section filled by priming or flushing with priming fluid by operating personnel and/or ascertaining this connection,
request a connection of the venous shunt section to the venous section filled by priming or flushing with priming fluid by operating personnel and/or ascertaining this connection, and
at least one of detect, quantify or check a recirculation and/or a recirculation rate at the shunt depending on the blood property and/or dialysate property recorded after the connection of the venous shunt section.

2. The extracorporeal blood treatment machine according to claim 1, wherein the extracorporeal blood treatment machine is configured to prime or flush the extracorporeal blood circuit with the priming fluid from the arterial section to the venous section by activating the blood pump via the control unit.

3. The extracorporeal blood treatment machine according to claim 1, wherein the extracorporeal blood treatment machine is configured to request the connection of the arterial shunt section to the arterial section filled by priming or flushing with priming fluid by operating personnel and/or ascertaining this connection based on a sensor signal or an input signal.

4. The extracorporeal blood treatment machine according to claim 1, wherein the extracorporeal blood treatment machine is configured to request the connection of the venous shunt section to the venous section filled by priming or flushing with priming fluid by operating personnel and/or ascertaining this connection based on a sensor signal or an input signal

5. The extracorporeal blood treatment machine according to claim 1, wherein the control unit is configured to detect the recirculation at the shunt at least depending on a deviation of a blood property recorded after the connection of the venous shunt section from a reference blood property without recirculation, and/or a value ratio of the blood property recorded after the connection of the venous shunt section to a reference blood property without recirculation, and/or an extremum or saddle point of the blood property recorded after the connection of the venous shunt section, and/or a gradient of the blood property recorded after the connection of the venous shunt section.

6. The extracorporeal blood treatment machine according to claim 5, wherein the control unit is configured to set a recirculation rate at the shunt at least depending on the deviation, and/or the value ratio of the blood properties recorded after connecting the venous shunt section to the reference blood properties without recirculation, and/or the gradient.

7. The extracorporeal blood treatment machine according to claim 1, wherein the control unit is configured to store the blood property at least at a first point in time and at a second point in time, and to detect recirculation and/or quantify the recirculation rate based on the blood property.

8. The extracorporeal blood treatment machine according to claim 1, wherein the control unit is configured to issue the request for connection of the venous shunt section and for connection of the arterial shunt section during the same interruption of flushing.

9. The extracorporeal blood treatment machine according to claim 1, wherein the control unit is configured to first issue a request to connect an arterial shunt ingate and, when connection of the arterial shunt ingate is ascertained, to control the conveying of the priming fluid via the venous shunt section into a waste container by activating the blood pump so that the connection of the venous shunt section takes place at a later interruption of the flushing than the connection of the arterial shunt section.

10. The extracorporeal blood treatment machine according to claim 9, wherein the control unit is configured to receive a signal from a second recording unit of the extracorporeal blood treatment machine from blood recorded at the venous section, to stop the blood pump and to issue the request for connection of the venous shunt ingate.

11. The extracorporeal blood treatment machine according to claim 1, wherein the control unit is configured to at least one of detect, quantify or check the recirculation at the shunt and/or the recirculation rate at the shunt at least depending on a deviation of the dialysate property recorded after the venous shunt section has been connected from a reference dialysate property without recirculation, and/or a value ratio of the dialysate property recorded after the venous shunt section has been connected to a reference dialysate property without recirculation, and/or a gradient of the dialysate property recorded after the venous shunt section has been connected.

12. A computer-implemented detection method for detecting recirculation at a shunt of an extracorporeal blood circuit of a blood treatment machine, in preparation for extracorporeal blood treatment, the extracorporeal blood treatment machine having a dialyzer with a semipermeable membrane at which blood from the extracorporeal blood circuit is brought into mass exchange with a dialysis fluid of a dialysis fluid circuit, the computer-implemented detection method comprising the steps of:
priming or flushing the extracorporeal blood circuit from its arterial section, which is intended for connection to an arterial shunt section, to its venous section, which is intended for connection to a venous shunt section, with a priming fluid;
requesting and/or ascertaining a connection of the arterial shunt section with the arterial section filled by priming or flushing with priming fluid;
requesting and/or ascertaining a connection of the venous shunt section with the venous section filled by priming or flushing with priming fluid;
recording a blood property by a blood property recording unit of the extracorporeal blood treatment machine and/or recording a dialysate property at a dialysis fluid outlet of the dialyzer by a dialysate property recording unit of the extracorporeal blood treatment machine, and
detecting and/or quantifying and/or checking the recirculation at the shunt at least depending on the blood property and/or dialysate property recorded after the venous shunt section has been connected.

13. The computer-implemented detection method according to claim 12, wherein the detection of recirculation at the shunt is performed at least depending on a deviation of the blood property recorded after connecting the venous shunt section to a reference blood property without recirculation, and/or a value ratio of the blood property recorded after connecting the venous shunt section to a reference blood property without recirculation, and/or an extremum or saddle point of the blood property recorded after connecting the venous shunt section, and/or a gradient of the blood property recorded after connecting the venous shunt section.

14. The computer-implemented detection method according to claim 13, further comprising the step of quantifying a recirculation rate at the shunt at least depending on the deviation, and/or the value ratio of the blood property recorded after connecting the venous shunt section to the reference blood property without recirculation, and/or the gradient, by the control unit.

15. The computer-implemented detection method according to claim 13, wherein the steps of connecting the venous shunt section and connecting the arterial shunt section are performed during the same interruption of flushing, or that between the step of connecting the arterial shunt ingate and the step of connecting the venous shunt section there is a step of conveying the priming fluid via the venous section into a waste container, so that the steps of connecting the venous shunt section and connecting the arterial shunt section take place during different interruptions of the flushing.

16. The computer-implemented detection method according to claim 12, wherein the step of recording the blood property comprises recording the blood property at the arterial section, the recording taking place at least at a first point in time and at a later second point in time.

17. The computer-implemented detection method according to claim 12, wherein the detection of the recirculation at the shunt is at least depending on a deviation of the dialysate property recorded after the connection of the venous shunt section from a reference dialysate property without recirculation, and/or a value ratio of the dialysate property recorded after the venous shunt section has been connected to a reference dialysate property without recirculation, and/or a gradient of the dialysate property recorded after the venous shunt section has been connected.

18. A computer program comprising instructions which, when executed by a computer, cause the computer to execute the computer-implemented detection method according to claim 12.
